(19) United States
(12) Patent Application Publication
(10) Pub. No.: US 2024/0304744 A1
(43) Pub. Date: Sep. 12, 2024

(54) RADIATION DETECTION ELEMENT, RADIATION DETECTION APPARATUS, X-RAY CT APPARATUS, AND MANUFACTURING METHOD OF RADIATION DETECTION ELEMENT

(71) Applicant: CANON KABUSHIKI KAISHA, Tokyo (JP)

(72) Inventors: TATSUYA RYOKI, Kanagawa (JP); TAKANORI YAMASHITA, Tokyo (JP); MASANOBU OHMURA, Kanagawa (JP)

(21) Appl. No.: 18/316,379

(22) Filed: May 12, 2023

(30) Foreign Application Priority Data
Mar. 7, 2023 (JP) 2023-034571

(51) Int. Cl. H01L 31/115; G01T 1/24; G01T 1/29

(52) U.S. Cl. CPC H01L 31/115; G01T 1/241; G01T 1/2928

(57) ABSTRACT

A radiation detection element according to the present invention includes: a single-crystal semiconductor substrate configured to convert incident radiation into an electric charge; a first cathode electrode provided on a first main surface of the single-crystal semiconductor substrate, the first cathode electrode having a first thickness; a second cathode electrode provided so as to face a side surface of the single-crystal semiconductor substrate, the second cathode electrode having a second thickness that is smaller than the first thickness; and an anode electrode provided on a second main surface of the single-crystal semiconductor substrate, the second main surface being on an opposite side of the first main surface.

### BACKGROUND OF THE INVENTION

### Field of the Invention

The present invention relates to a radiation detection element, a radiation detection apparatus, an X-ray CT apparatus, and a manufacturing method of the radiation detection element.

### Description of the Related Art

An X-ray detection method in which X-rays are indirectly detected has been proposed. In this method, X-rays are incident on a scintillator (phosphor) to be converted into visible light, and the converted visible light is incident on a single-crystal semiconductor substrate so as to detect the X-rays. In addition to the above method, there has been proposed another X-ray detection method in which X-rays are directly incident on a single-crystal semiconductor substrate to be detected. In the former method, the sensitivity of the X-ray detection is degraded due to the conversion from X-rays to visible light, whereas in the latter method, since such conversion is not performed, highly sensitive X-ray detection can be expected. Hereinafter, the latter method will be referred to as a “direct detection type”.

A direct-detection-type radiation detection element is provided with a cathode electrode on a first main surface of a single-crystal semiconductor substrate and an anode electrode on a second main surface (a surface on an opposite side of the first main surface) of the single-crystal semiconductor substrate. The single-crystal semiconductor substrate converts incident radiation (such as X-rays and gamma rays) into an electric charge. The electric charge generated in the single-crystal semiconductor substrate can be collected by applying a voltage between the cathode electrode and the anode electrode to form an electric field. When the electric field is formed, since the electric field is weakened at a side-surface portion of the single-crystal semiconductor substrate, charge collection efficiency decreases (charge loss increases).

U.S. Patent Application Publication No. 2007/0194243 (Specification) discloses a technique for improving charge collection efficiency by providing a cathode electrode also on a side surface of a s single-crystal semiconductor substrate.

### SUMMARY OF THE INVENTION

A radiation detection element according to the present invention includes: a single-crystal semiconductor substrate configured to convert incident radiation into an electric charge; a first cathode electrode provided on a first main surface of the single-crystal semiconductor substrate, the first cathode electrode having a first thickness; a second cathode electrode provided so as to face a side surface of the single-crystal semiconductor substrate, the second cathode electrode having a second thickness that is smaller than the first thickness; and an anode electrode provided on a second main surface of the single-crystal semiconductor substrate, the second main surface being on an opposite side of the first main surface.

A first radiation detection apparatus according to the present invention includes the radiation detection element according to the present invention, the radiation detection element being provided in plurality and arranged in a planar manner. A second radiation detection apparatus according to the present invention includes: a plurality of radiation detection elements that include the radiation detection element according to the present invention and a radiation detection element not having the second cathode electrode, the plurality of radiation detection elements being arranged without intervals in a first direction and in a second direction perpendicular to the first direction, wherein, among the plurality of radiation detection elements, the radiation detection element according to the present invention is arranged as an outermost radiation detection element, and the radiation detection element according to the present invention does not have the second cathode electrode on a side surface adjacent to another radiation detection element among side surfaces of the single-crystal semiconductor substrate and has the second cathode electrode on a side surface not adjacent to another radiation detection element among the side surfaces of the single-crystal semiconductor substrate.

A manufacturing method of a radiation detection element according to the present invention includes: a step of forming a first cathode electrode on a first main surface of a single-crystal semiconductor substrate that converts incident radiation into an electric charge, the first cathode electrode having a first thickness; a step of forming a second cathode electrode so as to face a side surface of the single-crystal semiconductor substrate, the second cathode electrode having a second thickness that is smaller than the first thickness; and a step of forming an anode electrode on a second main surface of the single-crystal semiconductor substrate, the second main surface being on an opposite side of the first main surface.

An X-ray computed tomography apparatus according to the present invention includes: an X-ray generation unit; the radiation detection apparatus according to the present invention configured to detect X-rays emitted from the X-ray generation unit; and a signal processing unit configured to process a signal output from the radiation detection apparatus.

Further features of the present invention will become apparent from the following description of exemplary embodiments with reference to the attached drawings.

### BRIEF DESCRIPTION OF THE DRAWINGS

FIGS. 1A to 1C illustrate configuration examples of a radiation detection element;

FIGS. 2A to 2C illustrate modification examples of the radiation detection element;

FIGS. 3A to 3G illustrate examples of a plurality of radiation detection elements that are tiled; and

FIG. 4 is a block diagram illustrating an X-ray computed tomography (CT) apparatus.

### DESCRIPTION OF THE EMBODIMENTS

If the technique disclosed in U.S. Patent Application Publication No. 2007/0194243 (Specification) is used, the size of the radiation detection element increases, which results in decreasing resolving power (resolution) in a case where a plurality of radiation detection elements are arranged (tiled) (decreasing the number of radiation detection elements that can be arranged in a certain area).

The present disclose provides a technique for improving resolution in a case where a plurality of radiation detection elements are arranged, while preventing a decrease in charge collection efficiency.

Hereinafter, an embodiment of the present invention will be described. A radiation detection element according to the present embodiment is an element (chip) that employs a method in which radiation such as X-rays or gamma rays is directly incident on a single-crystal semiconductor substrate to be detected. Hereinafter, this method will be referred to as a “direct detection type”. The single-crystal semiconductor substrate of the direct-detection-type radiation detection element is formed of, for example, a single crystal of a cadmium zinc telluride (CdZnTe: Cd₁₋ₓZnₓTe (x is, for example, 0.5 or less)) semiconductor, which is an alloy of cadmium telluride CdTe and zinc telluride ZnTe. A Cd₁₋ₓZnₓTe semiconductor is also referred to as CZT. In the present embodiment, CZT will be mainly described. However, the present invention is not limited to this embodiment and can be applied to any single-crystal semiconductor substrate capable of directly detecting X-rays. For example, the present invention can be applied to a single-crystal semiconductor substrate that includes cadmium telluride CdTe, cadmium tungstate CdWO₄, sodium iodide Nal, cesium iodide CsI, or the like.

### Radiation Detection Element

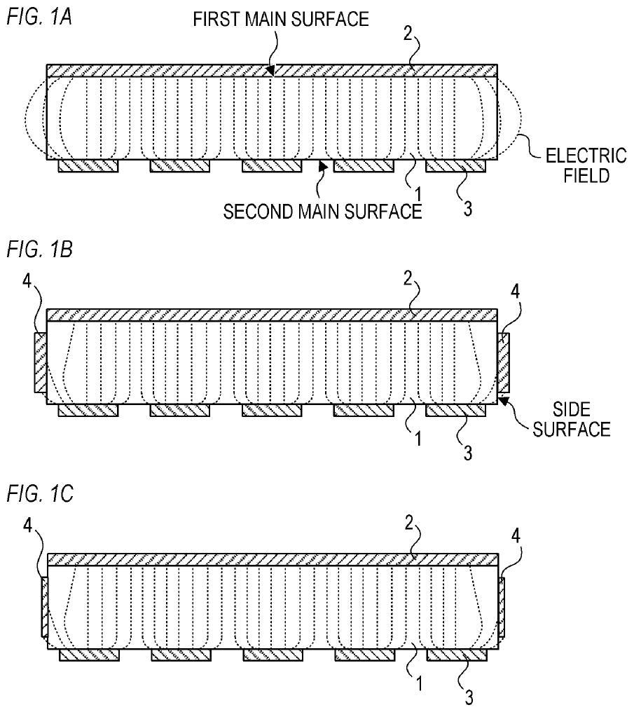

FIG. 1A is a sectional view illustrating a configuration example of a direct-detection-type radiation detection element, taken along a plane perpendicular to a main surface (a first main surface or a second main surface, which will be described below) of a single-crystal semiconductor substrate. As illustrated in FIG. 1A, the direct-detection-type radiation detection element is provided with a cathode electrode 2 on a first main surface of a single-crystal semiconductor substrate 1 and an anode electrode 3 on a second main surface (a surface on an opposite side of the first main surface) of the single-crystal semiconductor substrate 1. The anode electrode 3 is provided for each pixel, and in FIG. 1A, a plurality of anode electrodes 3 are provided in a single radiation detection element so that a plurality of pixels are arranged in the single radiation detection element. The single-crystal semiconductor substrate 1 converts incident radiation into an electric charge. The electric charge generated in the single-crystal semiconductor substrate 1 can be collected by applying a voltage between the cathode electrode 2 and the anode electrode 3 to form an electric field. When the electric field is formed, since the electric field is weakened at a side-surface portion of the single-crystal semiconductor substrate 1, charge collection efficiency decreases (charge loss increases).

In view of the above-described problem, a technique for improving the charge collection efficiency has been proposed. In this technique, as illustrated in FIG. 1B, a cathode electrode 4 having the same thickness as that of the cathode electrode 2 on the first main surface is provided on an individual side surface of the single-crystal semiconductor substrate 1. The potential of the cathode electrode 4 is set to, for example, the same potential as that of the cathode electrode 2.

However, the configuration illustrated in FIG. 1B increases the size of the radiation detection element, which results in decreasing resolving power (resolution) in a case where a plurality of radiation detection elements are arranged (tiled) (decreasing the number of radiation detection elements that can be arranged in a certain area).

Therefore, in the present embodiment, as illustrated in FIG. 1C, the cathode electrode 4 on the side surface is made thinner than the cathode electrode 2 on the first main surface. With this configuration, the increase in size of the radiation detection element can be prevented. Consequently, it is possible to increase the resolution in a case where a plurality of radiation detection elements are arranged, while preventing a decrease in charge collection efficiency.

### Manufacturing Method of Radiation Detection Element

The radiation detection element according to the present embodiment is produced by a manufacturing method including the following three steps, for example. In the first step, the cathode electrode 2 having a first thickness is formed on the first main surface of the single-crystal semiconductor substrate 1. In the second step, the cathode electrode 4 having a second thickness is formed on a side surface of the single-crystal semiconductor substrate 1. The second thickness is at least smaller than the first thickness. In the third step, the anode electrode 3 is formed on the second main surface of the single-crystal semiconductor substrate 1.

Each of the above-described three steps includes, for example, a step of forming an electrode layer on the single-crystal semiconductor substrate 1 by sputtering, a step of masking the electrode layer with a resist, a step of etching the electrode layer, and a step of removing the resist. Nickel, gold, platinum, indium, nickel/gold alloy, titanium/tungsten alloy, platinum/gold alloy, and the like can be used for the various electrodes. The thickness of each electrode is determined by, for example, the etching time and the etchant. For example, the cathode electrode 2 on the first main surface is formed to be approximately 20 nm to 250 nm thick, and the cathode electrode 4 on the side surface is formed to be thinner than the cathode electrode 2. The anode electrode 3 on the second main surface is formed to have the same thickness as that of the cathode electrode 2 on the first main surface, for example.

### Modifications of Radiation Detection Element

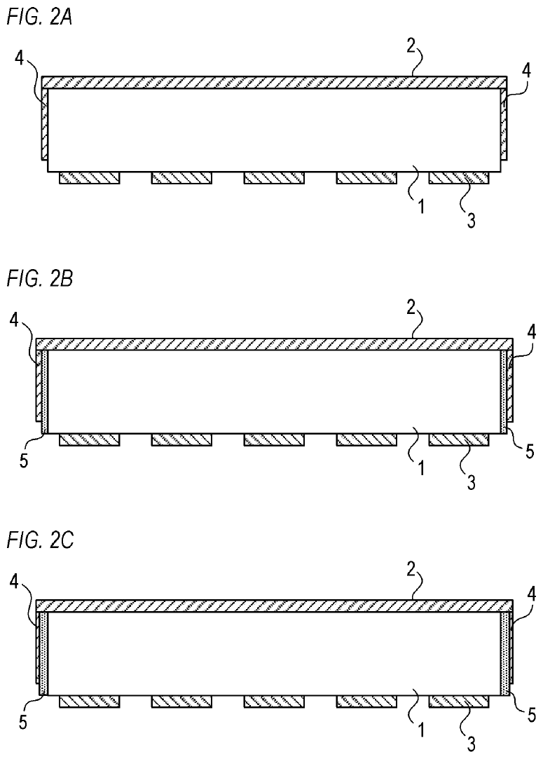

The configuration of the radiation detection element according to the present embodiment may be modified from the configuration illustrated in FIG. 1C to configurations illustrated in FIGS. 2A to 2C. In the configurations illustrated in FIGS. 2A to 2C, too, the cathode electrode 4 on the side surface is thinner than the cathode electrode 2 on the first main surface.

In FIG. 2A, the cathode electrode 2 on the first main surface and the cathode electrode 4 on the side surface are integrated. With this configuration, the cathode electrode 4 faces the side surface of the single-crystal semiconductor substrate 1 over a wider area than that in the configuration illustrated in FIG. 1C. Consequently, the configuration illustrated in FIG. 2A can further prevent the decrease in charge collection efficiency when compared to the configuration illustrated in FIG. 1C. The configuration illustrate in FIG. 2A can also be regarded as a configuration in which the cathode electrode 2 on the first surface is extended to the side surface of the single-crystal semiconductor substrate 1.

In FIG. 2B, an insulating layer 5 is provided between the side surface of the single-crystal semiconductor substrate 1 and the cathode electrode 4. The other portions in the configuration illustrated in FIG. 2B are the same as those illustrated in FIG. 2A. With this configuration, the withstand voltage between the anode electrode 3 on the second main surface and the cathode electrode 4 on the side surface can be improved to reduce leakage current. Consequently, the configuration illustrated in FIG. 2B can further prevent the decrease in charge collection efficiency when compared to the configuration illustrated in FIG. 2A. Alternatively, the insulating layer 5 may be added to the configuration illustrated in FIG. 1C.

In FIG. 2C, while a sum of the thickness of the cathode electrode 4 and the thickness of the insulating layer 5 remains the same as the configuration (a predetermined value) in FIG. 2B, the cathode electrode 4 is made thinner than the configuration in FIG. 2B, and the insulating layer 5 is made thicker than the configuration in FIG. 2B. For example, the insulating layer 5 is made thicker than the cathode electrode 4. The other portions in the configuration illustrated in FIG. 2C are the same as those illustrated in FIG. 2B. With this configuration, the withstand voltage between the anode electrode 3 on the second main surface and the cathode electrode 4 on the side surface can be further improved to further reduce leakage current. Consequently, the configuration illustrated in FIG. 2C can further prevent the decrease in charge collection efficiency when compared to the configuration illustrated in FIG. 2B.

### Radiation Detection Apparatus

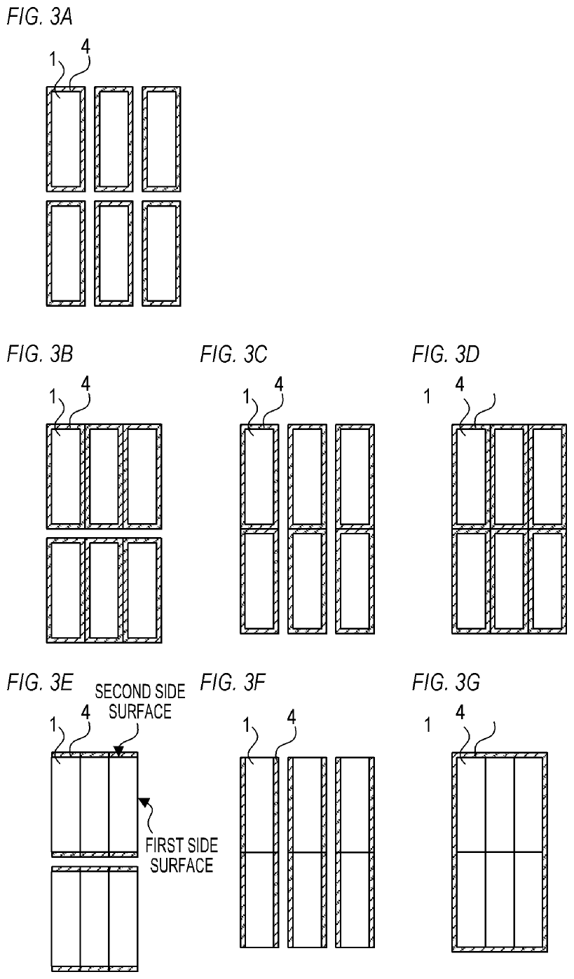

When a radiation detection apparatus is configured by using the radiation detection element according to the present embodiment, the radiation detection element provided in plurality are arranged in a planar manner (a plurality of radiation detection elements are tiled). FIG. 3A is a plan view illustrating an example of a radiation detection apparatus (a plurality of tiled radiation detection elements) viewed from a direction perpendicular to the main surfaces of the plurality of radiation detection elements. In FIG. 3A, only the single-crystal semiconductor substrates 1 and the cathode electrodes 4 on the side surfaces are illustrated. The arrangement pattern of the plurality of radiation detection elements is not particularly limited. In FIG. 3A, the plurality of radiation detection elements are arranged in a matrix (in a row direction and a column direction). In FIG. 3A, the plurality of radiation detection elements are arranged at intervals in both the row direction and the column direction.

### Modifications of Tiling

The configuration of the radiation detection apparatus according to the present embodiment may be modified from the configuration illustrated in FIG. 3A to configurations illustrated in FIGS. 3B to 3G.

In FIG. 3B, the plurality of radiation detection elements are arranged without intervals in the row direction and arranged at intervals in the column direction. In FIG. 3C, the plurality of radiation detection elements are arranged without intervals in the column direction and arranged at intervals in the row direction. With these configurations, it is possible to further increase the resolution in the case of tiling, when compared to the configuration (configuration illustrated in FIG. 3A) in which the plurality of radiation detection elements are arranged at intervals from each other.

In FIG. 3D, the plurality of radiation detection elements are arranged without intervals in both the row direction and the column direction. With this configuration, it is possible to further increase the resolution in the case of tiling, when compared to the configurations illustrated in FIGS. 3B and 3C.

The single-crystal semiconductor substrate 1 has two first side surfaces, which are side surfaces perpendicular to the row direction (side surfaces parallel to the column direction) and two second side surfaces, which are side surfaces perpendicular to the column direction (side surfaces parallel to the row direction). In FIG. 3E, while the cathode electrodes 4 are provided on the second side surfaces, the cathode electrodes 4 are not provided on the first side surfaces. The other portions in the configuration illustrated in FIG. 3E are the same as those illustrated in FIG. 3B. By not providing the cathode electrodes 4 on the first side surfaces and having no (zero) intervals between the plurality of radiation detection elements in the row direction, the configuration illustrated in FIG. 3E can further increase the resolution in the case of tiling, when compared to the configuration illustrated in FIG. 3B. In FIG. 3E, the cathode electrodes 4 are not provided on the outermost first side surfaces, and this results in weakening the electric fields of these portions. Thus, the cathode electrodes 4 may be provided on the outermost first side surfaces. The electric fields of the other first-side-surface portions are not easily weakened even if the cathode electrodes 4 are not provided thereto.

In FIG. 3F, while the cathode electrodes 4 are provided on the first side surfaces, the cathode electrodes 4 are not provided on the second side surfaces. The other portions in the configuration illustrated in FIG. 3F are the same as those illustrated in FIG. 3C. By not providing the cathode electrodes 4 on the second side surfaces and having no (zero) intervals between the plurality of radiation detection elements in the column direction, the configuration illustrated in FIG. 3F can further increase the resolution in the case of tiling, when compared to the configuration illustrated in FIG. 3C. In FIG. 3F, the cathode electrodes 4 are not provided on the outermost second side surfaces, and this results in weakening the electric fields of these portions. Thus, the cathode electrodes 4 may be provided on the outermost second side surfaces. The electric fields of the other second-side-surface portions are not easily weakened even if the cathode electrodes 4 are not provided thereto.

In FIG. 3G, no cathode electrodes 4 are basically provided on the side surfaces of the single-crystal semiconductor substrate 1. The other portions in the configuration illustrated in FIG. 3G are the same as those illustrated in FIG. 3D. By not providing the cathode electrodes 4 on the side surfaces of the single-crystal semiconductor substrate 1 and having no (zero) intervals between the plurality of radiation detection elements in both the row direction and the column direction, it is possible to further increase the resolution in the case of tiling, when compared to the configuration illustrated in FIG. 3D. However, if the cathode electrodes 4 are not provided on any of the side surfaces, the electric fields are weakened at the portions of the outermost side surfaces (side surfaces not adjacent to other radiation detection elements) among the plurality of side surfaces of the plurality of single-crystal semiconductor substrates 1. Thus, in FIG. 3G, the cathode electrodes 4 are provided on the outermost side surfaces to prevent the electric fields from being weakened (to prevent the decrease in charge collection efficiency). The cathode electrodes 4 may be provided on all the outermost side surfaces or may be provided on only a part of the outermost side surfaces.

### X-Ray CT Apparatus

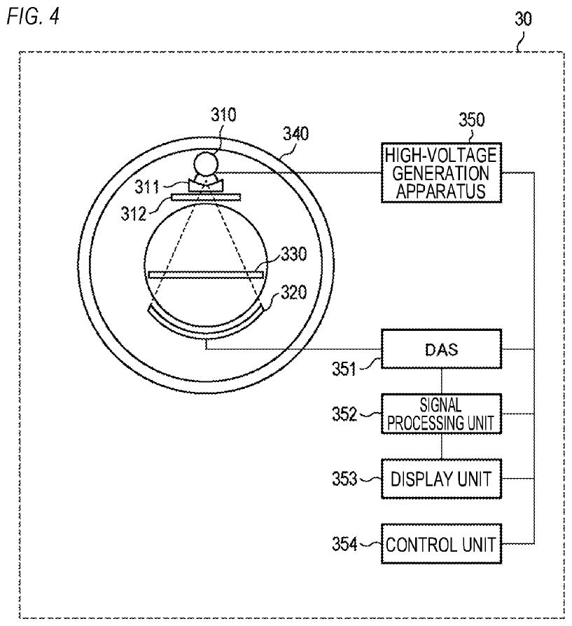

The radiation detection apparatuses according to the present embodiment can be applied to a detector of an X-ray CT apparatus. FIG. 4 is a block diagram illustrating an X-ray CT apparatus according to the present embodiment. An X-ray CT apparatus 30 according to the present embodiment includes an X-ray generation unit 310, a wedge 311, a collimator 312, an X-ray detection unit 320, a top plate 330, a rotating frame 340, a high-voltage generation apparatus 350, a data acquisition system (DAS) 351, a signal processing unit 352, a display unit 353, and a control unit 354.

The X-ray generation unit 310 includes, for example, a vacuum tube that generates X-rays. A high voltage and a filament current are supplied from the high-voltage generation apparatus 350 to the vacuum tube of the X-ray generation unit 310. X-rays are generated by irradiation of thermal electrons from a cathode (filament) toward an anode (target).

The wedge 311 is a filter that adjusts the amount of X-rays emitted from the X-ray generation unit 310. The wedge 311 attenuates the amount of X-rays so that the X-rays emitted from the X-ray generation unit 310 to an object have a predetermined distribution. The collimator 312 includes a lead plate or the like that narrows the irradiation range of the X-rays that have passed through the wedge 311. The X-rays generated by the X-ray generation unit 310 are shaped into a cone beam shape via the collimator 312 and reach the object on the top plate 330.

The X-ray detection unit 320 is configured using the radiation detection apparatus according to the present embodiment. The X-ray detection unit 320 detects X-rays that have been emitted from the X-ray generation unit 310 and passed through the object and outputs a signal corresponding to the amount of the X-rays to the DAS 351.

The rotating frame 340 has an annular shape and is configured to be rotatable. The X-ray generation unit 310 (the wedge 311, the collimator 312) and the X-ray detection unit 320 are arranged to face each other inside the rotating frame 340. The X-ray generation unit 310 and the X-ray detection unit 320 are rotatable together with the rotating frame 340.

The high-voltage generation apparatus 350 includes a booster circuit and outputs a high voltage to the X-ray generation unit 310. The DAS 351 includes an amplifier circuit and an A/D conversion circuit and outputs a signal from the X-ray detection unit 320 to the signal processing unit 352 as a digital signal.

The signal processing unit 352 includes a central processing unit (CPU), a read-only memory (ROM), and a random access memory (RAM) and is capable of performing image processing and the like on digital data. The display unit 353 includes a flat display device or the like and can display an X-ray image. The control unit 354 includes a CPU, a ROM, a RAM, and the like and controls the entire operation of the X-ray CT apparatus 30.

The embodiments (including the modifications) described above are merely examples, and configurations obtained by appropriately modifying or changing the above-described configurations within the scope of the gist of the present invention are also included in the present invention. Configurations obtained by appropriately combining the above-described configurations are also included in the present invention.

According to the present embodiment, it is possible to increase the resolution in a case where a plurality of radiation detection elements are arranged, while preventing a decrease in charge collection efficiency.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-034571, filed on Mar. 7, 2023, which is hereby incorporated by reference herein in its entirety.

What is claimed is:

1. A radiation detection element comprising:
a single-crystal semiconductor substrate configured to convert incident radiation into an electric charge;
a first cathode electrode provided on a first main surface of the single-crystal semiconductor substrate, the first cathode electrode having a first thickness;
a second cathode electrode provided so as to face a side surface of the single-crystal semiconductor substrate, the second cathode electrode having a second thickness that is smaller than the first thickness; and
an anode electrode provided on a second main surface of the single-crystal semiconductor substrate, the second main surface being on an opposite side of the first main surface.

2. The radiation detection element according to claim 1, wherein the first cathode electrode and the second cathode electrode are integrated.

3. The radiation detection element according to claim 1, further comprising an insulating layer provided between the side surface of the single-crystal semiconductor substrate and the second cathode electrode.

4. The radiation detection element according to claim 3, wherein the second cathode electrode and the insulating layer have respective thicknesses that are determined such that a sum of the thickness of the second cathode electrode and the thickness of the insulating layer is a predetermined value and that the insulating layer is thicker than the second cathode electrode.

5. The radiation detection element according to claim 1, wherein the single-crystal semiconductor substrate includes cadmium telluride.

6. The radiation detection element according to claim 1, wherein the single-crystal semiconductor substrate includes cadmium zinc telluride.

7. The radiation detection element according to claim 1, wherein the single-crystal semiconductor substrate includes any of cadmium tungstate, sodium iodide, and cesium iodide.

8. A radiation detection apparatus comprising the radiation detection element according to claim 1, the radiation detection element being provided in plurality and arranged in a planar manner.

9. The radiation detection apparatus according to claim 8, wherein the plurality of radiation detection elements are arranged without intervals in a first direction and in a second direction perpendicular to the first direction.

10. The radiation detection apparatus according to claim 8, wherein the plurality of radiation detection elements are arranged without intervals in a first direction and at intervals in a second direction perpendicular to the first direction.

11. A radiation detection apparatus comprising:
a plurality of radiation detection elements that include the radiation detection element according to claim 1 and a radiation detection element not having the second cathode electrode, the plurality of radiation detection elements being arranged without intervals in a first direction and in a second direction perpendicular to the first direction,
wherein, among the plurality of radiation detection elements, the radiation detection element according to claim 1 is arranged as an outermost radiation detection element, and
the radiation detection element according to claim 1 does not have the second cathode electrode on a side surface adjacent to another radiation detection element among side surfaces of the single-crystal semiconductor substrate and has the second cathode electrode on a side surface not adjacent to another radiation detection element among the side surfaces of the single-crystal semiconductor substrate.

12. The radiation detection apparatus according to claim 10, wherein
the single-crystal semiconductor substrate has two first side surfaces perpendicular to the first direction and two second side surfaces perpendicular to the second direction, and
the radiation detection element according to claim 1 has no cathode electrodes facing the first side surfaces and has the second cathode electrodes facing the second side surfaces.

13. A manufacturing method of a radiation detection element, the manufacturing method comprising:
a step of forming a first cathode electrode on a first main surface of a single-crystal semiconductor substrate that converts incident radiation into an electric charge, the first cathode electrode having a first thickness;
a step of forming a second cathode electrode so as to face a side surface of the single-crystal semiconductor substrate, the second cathode electrode having a second thickness that is smaller than the first thickness; and
a step of forming an anode electrode on a second main surface of the single-crystal semiconductor substrate, the second main surface being on an opposite side of the first main surface.

14. The manufacturing method of the radiation detection element according to claim 13, wherein the single-crystal semiconductor substrate includes cadmium telluride.

15. The manufacturing method of the radiation detection element according to claim 13, wherein the single-crystal semiconductor substrate includes cadmium zinc telluride.

16. The manufacturing method of the radiation detection element according to claim 13, wherein the single-crystal semiconductor substrate includes any of cadmium tungstate, sodium iodide, and cesium iodide.

17. An X-ray computed tomography apparatus comprising:
an X-ray generation unit;
the radiation detection apparatus according to claim 8 configured to detect X-rays emitted from the X-ray generation unit; and
a signal processing unit configured to process a signal output from the radiation detection apparatus.
